(19) United States
(12) Patent Application Publication
(10) Pub. No.: US 2025/0367644 A1
(43) Pub. Date: Dec. 4, 2025

(54) CATALYSTS SUITABLE FOR MAKING LIGHT OLEFINS BY DEHYDROGENATION THAT INCLUDE IRON

(71) Applicant: Dow Global Technologies LLC, Midland, MI (US)

(72) Inventors: Manish Sharma, Freeport, TX (US); Brian W. Goodfellow, Freeport, TX (US); Lin Luo, Freeport, TX (US); Andrzej Malek, Midland, MI (US)

(73) Assignee: Dow Global Technologies LLC, Midland, MI (US)

(21) Appl. No.: 18/874,232

(22) PCT Filed: Jun. 12, 2023
(86) PCT No.: PCT/US2023/068290

§ 371 Date: Dec. 12, 2024

Related U.S. Application Data
(60) Provisional application No. 63/352,019, filed on Jun. 14, 2022.

(51) Int. Cl. B01J 23/89; B01J 21/12; B01J 35/30; B01J 37/02; B01J 37/08

(52) U.S. Cl. CPC B01J 23/896; B01J 21/12; B01J 35/394; B01J 37/0205; B01J 37/0236; B01J 37/024; B01J 37/08

(57) ABSTRACT

A catalyst includes from 5 ppmw to 1000 ppmw of platinum, from 0.1 wt. % to 10 wt. % of gallium, from 2300 ppmw to 30000 ppmw of iron, and at least 85 wt. % support, wherein the support includes one or more of alumina, silica, or combinations thereof.

### CROSS REFERENCE TO RELATED APPLICATION

This application claims priority to U.S. Provisional Application Ser. No. 63/352,019, filed Jun. 14, 2022, which is hereby incorporated by reference in its entirety.

### TECHNICAL FIELD

Embodiments described herein generally relate to chemical processing and, more specifically, to catalysts and systems for light olefin production.

### BACKGROUND

Light olefins, such as propylene, may be used as base materials to produce many different materials, such as polypropylene, isopropanol, and acrylic acid, which may be used in, e.g., packaging, construction, and textiles. As a result of this utility, there is a worldwide demand for light olefins. Suitable processes for producing light olefins generally depend on the given chemical feed and include those that utilize fluidized catalysts. For example, light olefins may be formed by the catalytic dehydrogenation of alkanes in a fluidized bed reactor. However, there is a need for improvement in the systems and associated catalysts used to make light olefins.

### SUMMARY

Some methods and associated systems used to make light olefins utilize catalysts for the dehydrogenation of alkanes. Some such catalysts include platinum, gallium, and a support. It has been found that the addition of iron in such catalyst, in particular amounts, may lead to enhanced performance in light olefin production systems. More specifically, iron in amounts of from 2300 ppmw to 30000 ppmw may provide benefits such as enhanced combustion of supplemental fuels, such as methane, that may be utilized to heat the catalyst to a reaction temperature for the dehydrogenation reaction.

According to one or more embodiments of the present disclosure, a catalyst may comprise from 5 to 1000 ppmw of platinum, from 0.1 wt. % to 10 wt. % of gallium, from 2300 ppmw to 30000 ppmw of iron, and at least 85 wt. % support. The support may comprise one or more of alumina, silica, or combinations thereof.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawing and claims, or recognized by practicing the described embodiments. The drawing is included to provide a further understanding of the embodiments and, together with the detailed description, serves to explain the principles and operations of the claimed subject matter. However, the embodiment depicted in the drawing is illustrative and exemplary in nature, and not intended to limit the claimed subject matter.

### BRIEF DESCRIPTION OF THE DRAWINGS

The following detailed description may be better understood when read in conjunction with the following drawing, in which:

FIG. 1 schematically depicts a reactor system, according to one or more embodiments of the present disclosure.

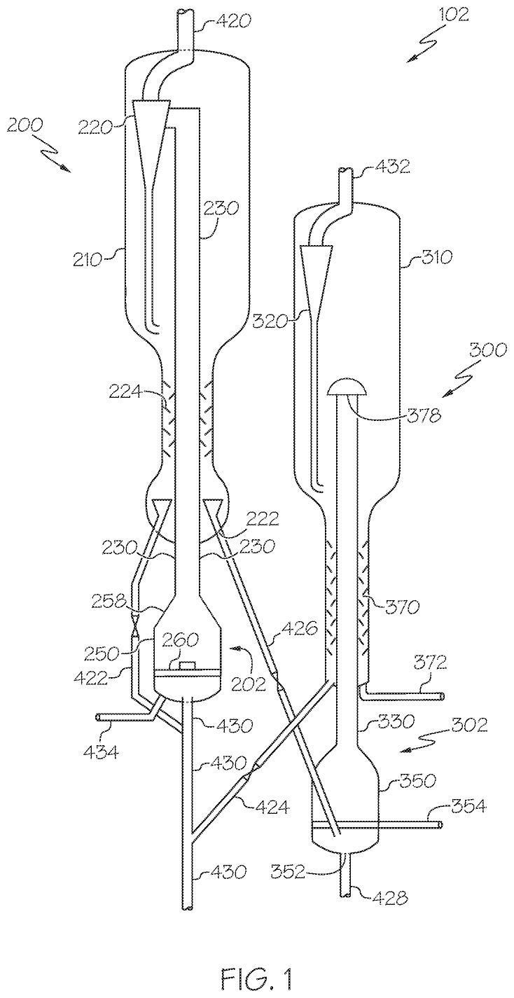

When describing the simplified schematic illustration of FIG. 1, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawing.

### DETAILED DESCRIPTION

The present disclosure is directed to catalysts suitable for making light olefins by dehydrogenation. For example, and as described herein, catalysts suitable for dehydrogenation may include from 0.1 wt. % to 10 wt. % of gallium, from 5 ppmw to 1000 ppmw platinum, from 2300 ppmw to 30000 ppmw of iron, and at least 85 wt. % support. In one or more embodiments, such catalysts provide dual catalytic functionality for dehydrogenation of alkanes as well as combustion of supplemental fuels. Such catalysts including iron may be particularly well suited for fluidized dehydrogenation of light alkanes to light olefins, such as propane to propylene, where methane is utilized as a supplemental fuel to heat the catalyst.

In one or more embodiments, the catalyst may comprise, consist essentially of, or consist of gallium, platinum, iron, and a support. As described herein, “consisting essentially of” refers to materials with less than 1 wt. % of the non-recited materials (i.e., consisting essentially of A and B means A and B combined are at least 99 wt. % of the composition). As is described herein, the catalyst may be solid particles suitable for fluidization.

In one or more embodiments, the catalyst may comprise gallium in an amount from 0.1 wt. % to 10 wt. % based on the total mass of the catalyst. Gallium may catalyze the dehydrogenation of alkanes to alkenes, particularly when used in combination with platinum. Such materials may additionally catalyze the combustion of coke and supplemental fuels. For example, the catalyst may comprise gallium in an amount from 0.1 wt. % to 0.25 wt. %, from 0.25 wt. % to 0.5 wt. %, from 0.5 wt. % to 0.75 wt. %, from 0.75 wt. % to 1 wt. %, from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, or any combination of these ranges. In some embodiments, the catalyst may comprise gallium in an amount from 0.1 wt. % to 9 wt. %, from 0.1 wt. % to 8 wt. %, from 0.1 wt. % to 7 wt. %, from 0.1 wt. % to 6 wt. %, or from 0.1 wt. % to 5 wt. %. Without being bound by theory, it is believed that compositions having gallium in an amount less than 0.1 wt. % negatively impacts the catalyst's ability to catalyze the alkane dehydrogenation process by lowering both the percentage of total alkane dehydrogenated and the percentage of dehydrogenated alkane that is the intended product. However, it is believed that compositions having gallium in an amount exceeding 10 wt. % may negatively impact the catalyst's ability to catalyze the alkane dehydrogenation process, negatively impact the catalyst's selectivity towards the intended product, or both.

In one or more embodiments, the catalyst may comprise platinum in an amount from 5 ppmw to 1000 ppmw based on the total mass of the catalyst. Platinum may catalyze the dehydrogenation of alkanes to alkenes, particularly when used in combination with gallium. Such materials may additionally catalyze the combustion of coke and supplemental fuels. For example, the catalyst may comprise platinum in an amount from 5 ppmw to 50 ppmw, from 50 ppmw to 100 ppmw, from 100 ppmw to 200 ppmw, from 200 ppmw to 300 ppmw, from 300 ppmw to 400 ppmw, from 400 ppmw to 500 ppmw, from 500 ppmw to 600 ppmw, from 600 ppmw to 700 ppmw, from 700 ppmw to 800 ppmw, from 800 ppmw to 900 ppmw, from 900 ppmw to 1000 ppmw, or any combination of these ranges. In some embodiments, the catalyst may comprise platinum in an amount from 5 ppmw to 900 ppmw, from 5 ppmw to 800 ppmw, from 5 ppmw to 600 ppmw, from 5 ppmw to 500 ppmw, or from 10 ppmw to 400 ppmw. Without being bound by theory, it is believed that compositions having platinum in an amount less than 5 ppmw negatively impacts the catalyst's ability to catalyze the alkane dehydrogenation process by lowering both the percentage of total alkane dehydrogenated and the percentage of dehydrogenated alkane that is the intended product. However, it is believed that compositions having platinum in an amount exceeding 1000 ppmw may negatively impact the catalyst's ability to catalyze the alkane dehydrogenation process, negatively impact the catalyst's selectivity towards the intended product, or both.

In one or more embodiments, the catalyst may comprise iron in an amount from 2300 ppmw to 30000 ppmw based on the total weight of the catalyst. The incorporation of iron may promote combustion of methane while not having significant impact on the dehydrogenation of alkanes. For example, the catalyst may comprise iron in an amount from 2300 ppmw to 3000ppmw, from 3000 ppmw to 4000 ppmw, from 4000 ppmw to 5000 ppmw, from 5000 ppmw to 7500 ppmw, from 7500 ppmw to 10000 ppmw, from 10000 ppmw to 15000 ppmw, from 15000 ppmw to 20000 ppmw, from 20000 ppmw to 25000 ppmw, from 25000 ppmw to 30000 ppmw, or any combination of these ranges. In some embodiments, the catalyst may comprise iron in an amount from 2300 ppmw to 25000 ppmw, from 2300 ppmw to 20000 ppmw, from 2300 ppmw to 15000 ppmw, from 2300 ppmw to 12500 ppmw, from 2300 ppmw to 10000 ppmw, or from 2300 ppmw to 8000 ppmw.

Without being bound by theory, it is believed that compositions having iron in an amount less than 2300 ppmw may not produce the desired improvement in the catalyst's methane combustion performance. Catalyst compositions having iron in an amount between 2300 ppmw and 30000 ppmw may have significantly improved methane combustion performance even when compared to compositions that contain iron, but in an amount less than 2300 ppmw. However, it is believed that while, compositions having iron in an amount exceeding 30000 ppmw may still have enhanced methane combustion performance, iron in an amount exceeding 30000 ppmw may negatively impact the catalyst's dehydrogenation performance by lowering both the percentage of total alkane that is dehydrogenated and the percentage of dehydrogenated alkane that is the intended product. The balance of performance between methane combustion and alkane dehydrogenation is ideal in compositions having iron in an amount from 2300 ppmw to 30000 ppmw. Without being bound by theory, it is further believed that compositions with iron loading below 2300 ppmw may suffer increased catalyst deactivation overtime during the dehydrogenation and fuel combustion process, when compared to catalyst compositions with iron loading greater than or equal to 2300 ppmw.

As is described herein, in one or more embodiments, the catalyst may comprise a support. The support may comprise one or more of alumina, silica, or combinations thereof. For example, in some embodiments the support may comprise one or more of alumina, silica-containing alumina, zirconia-containing alumina, titania-containing alumina, and lanthanum-containing alumina. The support may be present in an amount of at least 85 wt. % relative to the total weight of the catalyst, such as at least 85 wt. %, at least 90 wt. %, or at least 95 wt. %. In some embodiments, the support comprises less than or equal to 99.5 wt. % of the catalyst. Generally, the wt. % of the support may fill the remainder of the total catalyst not specified by other materials.

In one or more embodiments, the catalyst may optionally comprise one or more alkali metals, one or more alkaline earth metals, or both in an amount from 0.01 wt. % to 2.5 wt. % based on the total weight of the catalyst. For example, the catalyst may comprise one or more alkali metals, one or more alkaline earth metals, or both in an amount from 0.01 wt. % to 0.05wt. %, from 0.05 wt. % to 0.1 wt. %, from 0.1 wt. % to 0.2 wt. %, from 0.2 wt. % to 0.3 wt. %, from 0.3 wt. % to 0.4 wt. %, from 0.4 wt. % to 0.5 wt. %, from 0.5 wt. % to 0.6 wt. %, from 0.6 wt. % to 0.7 wt. %, from 0.7 wt. % to 0.8 wt. %, from 0.8 wt. % to 0.9 wt. %, from 0.9 wt. % to 1 wt. %, from 1 wt. % to 1.25 wt. %, from 1.25 wt. % to 1.5 wt. %, from 1.5 wt. % to 1.75 wt. %, from 1.75 wt. % to 2 wt. %, from 2 wt. % to 2.25 wt. %, from 2.25 wt. % to 2.5 wt. %, or any combination of these ranges. In some embodiments, the catalyst may comprise one or more alkali metals, one or more alkaline earth metals, or both from 0.01 wt. % to 0.75 wt. %, from 0.02 wt. % to 0.6 wt. %, from 0.03 wt. % to 0.5 wt. %, from 0.04 wt. % to 0.4 wt. %, or from 0.05 wt. % to 0.3 wt. %. In some embodiments, the one or more alkali metals or one or more alkaline earth metals may be potassium. Without being bound by theory, it is believed that compositions having alkali metals or alkaline earth metals in an amount less than 0.01 wt. % may cause the dehydrogenation process to produce undesired products. However, it is believed that compositions having alkali metals or alkaline earth metals in an amount exceeding 2.5 wt. % may reduce the catalyst's dehydrogenation activity.

In one or more embodiments, the catalyst may comprise, consist essentially of, or consist of gallium, platinum, iron and a support. For example, the catalyst may comprise, consist essentially of, or consist of from 0.1 wt. % to 10 wt. % of gallium; from 5 ppmw to 1000 ppmw of platinum; from 2300 ppmw to 30000 ppmw iron; and at least 85 wt. % of a support. In one exemplary embodiment, the catalyst may comprise, consist essentially of, or consist of from 0.1 wt. % to 5 wt. % of gallium; from 10 ppmw to 400 ppmw of platinum; from 2300 ppmw to 8000 ppmw iron, and at least 85 wt. % of a support.

In one or more embodiments, the catalyst may include solid particulates that are capable of fluidization. In some embodiments, the catalyst may exhibit properties known in the industry as “Geldart A” or “Geldart B” properties. Catalyst type may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973) 285-292, the disclosures of which are incorporated herein by reference in their entireties.

Geldart Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal dp; or as the <45 micrometers (μm) proportion is increased; or as pressure, temperature, viscosity, and density of the gas increase. In general, the particles may exhibit a small mean particle size and/or low particle density (<1.4 grams per cubic centimeter, g/cm³), fluidize easily, with smooth fluidization at low gas velocities, and may exhibit controlled bubbling with small bubbles at higher gas velocities.

Geldart Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size ( [symbol] ) of 40 μm< [symbol] <500 μm when the density (ρp) is 1.4<ρp<4 g/cm³.

In one or more embodiments, the catalyst may be prepared via incipient wetness impregnation also known as dry impregnation or capillary impregnation. For example, such a process is described in Marceau et al., Impregnation and Drying, Synthesis of Solid Catalysts 59 (2008), which is incorporated herein by reference in its entirety. For example, the support may be impregnated using metal precursors, then dried at temperatures less than 200° C., and then calcined at temperatures less than 800° C. to produce the catalyst. For example, suitable metal precursors may include nitrate or amine nitrate metal precursors. Additionally, other suitable metal precursors are contemplated herein, as would be known by those skilled in the art. In some embodiments, the method of making the catalyst may comprise impregnating the support with gallium, platinum, and iron; drying the support; and calcining the support, wherein the catalyst comprises from 0.1 wt. % to 10 wt. % of gallium, from 5 ppmw to 1000 ppmw of platinum, from 2300 ppmw to 30000 ppmw of iron, and at least 85 wt. % support.

In one or more embodiments, the catalyst may be prepared by incipient wetness sequential impregnation, where materials are impregnated in a specific order, either before or after drying and calcining. In incipient wetness sequential impregnation, the catalyst is first impregnated with one or more metal precursors, dried at temperatures less than 200° C., and then calcined at temperatures less than 800° C. The catalyst then undergoes a least one additional cycle of impregnation, drying, and calcining with an additional metal precursor to create a finished catalyst. In incipient wetness sequential impregnation, the metals added to the catalyst can be added in sequential order in successive impregnation cycles. In one or more embodiments, the support is sequentially impregnated with gallium and platinum and then with iron. For some embodiments, the method of making a catalyst may comprise impregnating the support with gallium and platinum, drying the support, calcining the support, impregnating the support with iron following the drying and calcining, and drying and calcining the support following the impregnation with iron, wherein the catalyst comprises from 0.1 wt. % to 10 wt. % of gallium, from 5 ppmw to 1000 ppmw of platinum, from 2300 ppmw to 30000 ppmw iron and at least 85 wt. % support. In additional embodiments, the method of making a catalyst may comprise impregnating the support with iron to create an iron impregnated support, drying the iron impregnated support, calcining the iron impregnated support, impregnating the iron impregnated support with gallium and platinum following the drying and calcining, and drying and calcining the iron impregnated support following the impregnation with gallium and platinum, wherein the catalyst comprises from 0.1 wt. % to 10 wt. % of gallium, from 5 ppmw to 1000 ppmw of platinum, from 2300 ppmw to 30000 ppmw of iron, and at least 85 wt. % support.

Incipient wetness sequential impregnation allows the support to be impregnated with metals in a sequential order where some metals may be impregnated onto the support before others. The order of impregnation can be therefore be altered as desired. Additionally, other suitable methods for making the catalysts described herein are contemplated, as would be known by those skilled in the art.

In one or more embodiments the catalysts described herein may be used in the reactor system of FIG. 1 operating as a fluidized dehydrogenation reactor system to produce light olefins, such as propylene. However, it should be understood that the principles disclosed and taught herein may be applicable to other systems which utilize different system components oriented in different ways. For example, the concepts described may be equally applied to other systems with alternate reactor units and regeneration units, such as those that operate under non-fluidized conditions or include downers rather than risers. It should be further understood that not all portions of FIG. 1 should be construed as essential to the claimed subject matter. Moreover, while the catalyst compositions in the appended claims are described herein in the context of FIG. 1, such recited compositions should be understood as adaptable to other systems, as would be understood by those skilled in the art.

Now referring to FIG. 1, an example reactor system 102 that may be suitable for use with the catalysts described herein is schematically depicted. The reactor system 102 generally comprises multiple system components, such as a reactor portion 200 and a catalyst processing portion 300. As described herein, “system components” refer to portions of the reactor system 102, such as reactors, separators, transfer lines, combinations thereof, and the like. As used herein in the context of FIG. 1, the reactor portion 200 generally refers to the portion of the reactor system 102 in which the major process reaction takes place (e.g., dehydrogenation) to form the olefin-containing effluent. A hydrocarbon-containing feed enters the reactor portion 200, is contacted with a catalyst, converted to an olefin-containing effluent (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a catalyst separation section 210, which serves to separate the catalyst from the olefin-containing effluent formed in the reactor 202. Also, as used herein, the catalyst processing portion 300 generally refers to the portion of the reactor system 102 where the catalyst is in some way processed, such as by combustion, to, e.g., improve catalytic activity by decoking and/or heating the catalyst. The catalyst processing portion 300 may comprise a combustor 350 and a riser 330, and may additionally comprise a catalyst separation section 310. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

Generally as is described herein, in embodiments illustrated in FIG. 1, catalyst is cycled between the reactor portion 200 and the catalyst processing portion 300. It should be understood that when “catalysts” are referred to herein, they may refer to solid materials that are catalytically active for a desired reaction. The terms “catalytic activity” and “catalyst activity” refer to the degree to which the catalyst is able to catalyze the reactions conducted in the reactor system 102. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Coke may form on the catalyst within the reactor portion 200. Reactivation (sometimes called “regeneration” herein) may remove the contaminant such as coke, raise the temperature of the catalyst, or both. In embodiments, deactivated catalyst may be reactivated by catalyst reactivation in the catalyst processing portion 300. The deactivated catalyst may be reactivated by, but not limited to, removing coke by combustion, oxidizing the catalyst, other reactivation process, or combinations thereof. In some embodiments, the catalyst may be heated during reactivation by combustion of a supplemental fuel, such as methane, ethane, propane, natural gas, or combinations thereof. The reactivated catalyst from the catalyst processing portion 300 is then passed back to the reactor portion 200.

As is disclosed herein, in one or more embodiments the supplemental fuel may comprise methane. For example the supplemental fuel may comprise an amount of methane greater than or equal to 1 mol. %, such as greater than or equal to 2 mol. %, greater than or equal to 3 mol. %, greater than or equal to 4 mol. %, or even greater than or equal to 5 mol. %. In some embodiments the supplemental fuel comprises methane in an amount no more than 10 mol. %. In some embodiments, the supplemental fuel may comprise methane in an amount greater than 10 mol. %, such as greater than 20 mol. %, greater than 30 mol. %, greater than 40 mol. %, greater than 50 mol. %, greater than 60 mol. %, greater than 70 mol. %, greater than 80 mol. %, greater than 90 mol. %, or even 100 mol. %. Catalysts with improved methane combustion activity, such as those described herein that include manganese, can better utilize methane as a supplemental fuel to facilitate re-heating of the catalyst. The catalyst is heated during regeneration to aid with regeneration and also because heated catalyst serves as a heat carrier to carry heat from the combustor 350 to the reactor portion 200 to facilitate the dehydrogenation reaction.

In non-limiting examples, the reactor system 102 described herein may be utilized to produce light olefins from a hydrocarbon-containing feed. According to one or more embodiments, the reaction may be a dehydrogenation reaction. According to such embodiments, the hydrocarbon-containing feed may comprise one or more of ethyl benzene, ethane, propane, n-butane, and i-butane. In one or more embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of ethyl benzene. In one or more embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon-containing feed may comprise at least 50wt. %, at least 60 wt. %, at least 70 wt. %, at least 80 wt. %, at least 90 wt. %, at least 95 wt. % or even at least 99 wt. % of n-butane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

As described with respect to FIG. 1, the hydrocarbon-containing feed may enter feed inlet 434 into the reactor 202, and the olefin-containing effluent may exit the reactor system 102 via pipe 420. According to one or more embodiments, the reactor system 102 may be operated by feeding a hydrocarbon-containing feed (e.g., in a feed stream) and a fluidized catalyst into the upstream reactor section 250. The hydrocarbon-containing feed contacts the catalyst in the upstream reactor section 250, and each flow upwardly into and through the downstream reactor section 230 to produce an olefin-containing effluent.

Now referring to FIG. 1 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 1). This catalyst may be somewhat deactivated, but may still, in some embodiments, be suitable for reaction in the upstream reactor section 250, particularly when used in combination with reactivated catalyst.

In one or more embodiments, the catalyst may have a residence time within the reactor portion 200 of less than or equal to 3 minutes. As the term is used herein, “residence time” refers to the average amount of time the catalyst or other specified material spends within the reactor portion 200. As it is an average, the amount of time the catalyst may spend within the reactor portion 200 during any given cycle may not be equal to the average, but over time will average out to be equal to about the residence time. In some embodiments, the catalyst may have a residence time within the reactor portion 200 of less than or equal to 2.5 min., less than or equal to 2 min., less than or equal to 1.5 min., less than or equal to 1 min., less than or equal to 0.5 min. or less than or equal to 0.1 min. Without being bound by theory, it is believed that catalyst residence time greater than 3 minutes may increase equipment costs without a matching increase in catalyst dehydrogenation performance. However, it is believed that catalyst residence time less than 0.1 minutes may not allow the catalyst to sufficiently catalyze the dehydrogenation reaction.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at above choking velocity.

According to embodiments, the olefin-containing effluent and the catalyst may be passed out of the downstream reactor section 230 to a separation device 220 in the catalyst separation section 210, where the catalyst is at least partially separated from the olefin-containing effluent, which is transported out of the catalyst separation section 210. According to one or more embodiments, following separation from vapors in the separation device 220, the catalyst may generally move through the stripper 224 to the catalyst outlet port 222 where the catalyst is transferred out of the reactor portion 200 via standpipe 426 and into the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the present disclosure.

Still referring to FIG. 1, the separated catalyst is passed from the catalyst separation section 210 to the combustor 350. In the combustor 350, the catalyst may be processed by, for example, combustion of coke with oxygen. For example, and without limitation, the catalyst may be de-coked and/or supplemental fuel may be combusted to heat the catalyst. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. The separated catalyst is then passed through the oxygen treatment zone 370 within the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the hydrocarbon-containing feed and olefin-containing effluent may be gaseous, and the catalyst may be fluidized particulate solid.

In embodiments where methane is used as supplemental fuel during catalyst processing it may be important to ensure that the catalyst adequately catalyzes methane combustion to improve catalyst processing and to ensure that the catalyst is sufficiently heated during processing to the temperatures required for alkane dehydrogenation. A catalyst with iron in an amount from 2300 ppmw to 30000 ppmw, when compared to a conventional catalyst with less than 2300 ppmw of iron or more than 30000 ppmw of iron, may have improved methane combustion activity without having inadequate alkane dehydrogenation performance.

Referring now to the catalyst processing portion 300, as depicted in FIG. 1, the combustor 350 of the catalyst processing portion 300 may include one or more lower reactor portion inlet ports 352 and may be in fluid communication with the riser 330. Oxygen-containing gas, such as air, may be passed through pipe 428 into the combustor 350. The combustor 350 may be in fluid communication with the catalyst separation section 210 via standpipe 426, which may supply spent catalyst from the reactor portion 200 to the catalyst processing portion 300 for regeneration. The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

As described herein, the catalyst may be heated in the catalyst processing portion 300 by combustion of supplemental fuels. Supplemental fuels may combust with oxygen to heat the catalyst, and supplemental fuels such as hydrogen, methane, ethane, propane, natural gas, or combinations thereof may be utilized. Without being bound by any theory, when methane is utilized in the supplemental fuel, catalysts as described herein that include iron may better catalyze the combustion of methane to heat the catalyst. Catalysts which do not contain iron, when methane is utilized in the supplemental fuel, may be deficient by not promoting heating of the catalyst to a temperature needed for dehydrogenation.

As described in one or more embodiments, following separation of flue gas from catalyst in the riser termination separator 378 and secondary separation device 320, treatment of the processed catalyst with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone may be bubbling bed type fluidization. The oxygen treatment zone 370 may include an oxygen-containing gas inlet 372, which may supply an oxygen-containing gas to the oxygen treatment zone 370 for oxygen treatment of the catalyst.

As is disclosed herein, in one or more embodiments the catalyst may be exposed to an oxygen-containing gas in oxygen treatment zone 370. For example, the catalyst may be exposed to an oxygen-containing gas for from 2 min. to 20 min., such as from 2 min. to 4 min., from 4 min. to 6 min., from 6 min. to 8 min., from 8 min. to 10 min., from 10 min. to 12 min., from 12 min. to 14 min., from 14 min. to 16 min., from 16 min. to 18 min., from 18 min. to 20 min., or any combination of these ranges. In some embodiments the catalyst may be exposed to an oxygen containing gas from 4 min. to 18 min., from 6 min. to 17 min., from 8 min. to 16 min., or from 10 min. to 15 min. Without being bound by theory, it is believed that exposure of the catalyst to an oxygen-containing gas for more than 20 minutes may increase equipment costs without a matching increase in catalyst regeneration efficiency. However, it is believed that oxygen-containing gas exposure for less than 2 minutes may lead to less efficient regeneration of the catalyst which may reduce the catalyst's dehydrogenation activity.

In one or more embodiments, the light olefins may be present in a “product stream” sometimes called an “olefin-containing effluent” and include light olefins. Such a stream exits the reactor system of FIG. 1 and may be subsequently processed. As used in the present disclosure, the term “light olefins” refers to one or more of ethylene, propylene, and butene. The term butene includes any isomers of butene, such as α-butylene, cis-β-butylene, trans-β-butylene, and isobutylene. In some embodiments, the olefin-containing effluent includes at least 25 wt. % light olefins based on the total weight of the olefin-containing effluent. For example, the olefin-containing effluent may include at least 35 wt. % light olefins, at least 45 wt. % light olefins, at least 55 wt. % light olefins, at least 65 wt. % light olefins, or at least 75 wt. % light olefins based on the total weight of the olefin-containing effluent. The olefin-containing effluent may further comprise unreacted components of the hydrocarbon-containing effluent, as well as other reaction products that are not considered light olefins. The light olefins may be separated from unreacted components in subsequent separation steps.

### EXAMPLES

The various embodiments of the present disclosure will be further clarified by the following examples. The examples are illustrative in nature and should not be understood to limit the subject matter of the present disclosure.

### Example 1

### Effect of Iron loading

In Example 1, 12 different samples of catalytically active particles (i.e., catalysts) were prepared and the effect of iron loading on catalytically active particles was observed. For the purposes of Example 1, the samples were prepared by first preparing microspheroidal alumina support by spray drying a mixture of hydrated alumina and Ludox® Silica and then heating the resulting spray dried particles at a temperature of at least 1000° C. sufficient to achieve particles with particle size ranging from 5 μm to 300 μm, pore volume of 0.20÷0.10 ml/g, surface area of 70 +20 m2/g, and silica content 2.5 +2.5 wt. %. The catalyst materials were prepared using an incipient wetness impregnation method to load the designated metal or metals to the support using nitrate or amine nitrate metal precursors followed by drying at a temperature less than 200° C., and then calcination at a temperature less than 800° C. The exact compositions of the samples are provided in Table 1.

The samples were tested at ambient pressure under conditions of 0.5 grams (g) of the sample was mixed with 1.0 g inert silicon carbide and loaded into a quartz reactor. The reaction combustion reactivation cycle was carried out for 10 break in cycles followed by dehydrogenation and regeneration testing cycles. The break in cycles were run by performing two steps: a dehydrogenation step where a dehydrogenation process was performed at 625° C. with weight hourly space velocity “WHSV” propane of 10 hr-1 and feed composition of 90% propane/10% nitrogen for 60 seconds; and a reactivation step where the catalyst was heated to 730° C. under 100% air with a flow rate of 50 standard cubic centimeters per minute (sccm) for 5 minutes. After the catalyst had been run through 10 break in cycles it was tested in the dehydrogenation and regeneration testing cycles. The dehydrogenation and regeneration testing cycles were run be performing three steps: a dehydrogenation step using the same conditions as the dehydrogenation step of the break in cycles where dehydrogenation performance data were collected at 30 seconds time on stream; a combustion step where combustion was performed at 730° C. under 2.5 mol % Methane/balance air with a total flow of 50 sccm and a WHSV of Methane of 0.1 hr⁻¹ for 3 minutes where combustion performance data were collected at 60 seconds time on stream; and a reactivation step where the samples were heated to 730° C. under 100% air with a flow rate of 50 sccm for 2 minutes. The dehydrogenation and combustion performances of the samples are reported in Table 1 after 25 cycles.

TABLE 1

| | | | | | Propane | Propylene | Methane |
|---|---|---|---|---|---|---|---|
| | Pt | Ga | K | Fe | Conversion | Selectivity | Conversion |
| Sample | [ppm] | [wt. %] | [wt. %] | [ppm] | [%] | [%] | [%] |
| A | 300 | 1.6 | 0.25 | 0 | 39.4 | 98.7 | 85.2 |
| B | 300 | 1.6 | 0.25 | 1000 | 48.2 | 99.1 | 94.8 |
| C | 300 | 1.6 | 0.25 | 1500 | 48.7 | 99.2 | 94.2 |
| 1 | 300 | 1.6 | 0.25 | 3000 | 46.1 | 99.0 | 98.2 |
| 2 | 300 | 1.6 | 0.25 | 5000 | 44.7 | 99.1 | 99.5 |
| 3 | 300 | 1.6 | 0.25 | 8000 | 33.6 | 98.2 | 100 |
| 4 | 300 | 1.6 | 0.25 | 10000 | 27.8 | 97.0 | 100 |
| D | 0 | 0 | 0.25 | 3000 | 1.7 | 62.6 | 89.0 |
| E | 0 | 0 | 0.25 | 30000 | 4.3 | 72.1 | 100 |
| F | 0 | 1.6 | 0.25 | 3000 | 6.2 | 92.3 | 91.7 |
| G | 300 | 0 | 0.25 | 3000 | 3.1 | 83.7 | 99.7 |
| H | 0 | 0 | 0 | 3000 | 2.4 | 80.0 | 91.8 |

Table 1 indicates that all samples with iron in an amount from 2300 ppmw to 30000 ppmw (i.e., Samples 1-4) have significantly improved methane conversion when compared to the comparative examples with iron in an amount less than 2300 ppmw iron such as Comparative Examples A-C. Some samples with iron in an amount from 2300 ppmw to 30000 ppmw have improved methane conversion, propane conversion, and propylene selectivity. For example, Comparative Example A (void of iron) has worse propane conversion, propylene selectivity and methane conversion than Samples 1 and 2 which are identical to Comparative Example A except for the presence of iron. Table I also indicates that samples with iron in an amount from 2300 ppmw to 300000 ppmw have significantly improved methane conversion even compared to comparative examples with iron, but in an amount less than 2300 ppmw such as Comparative Examples B and C. For example, Sample 1 with 3000 ppmw of iron converts a higher percentage of methane than Comparative Example C, which only has 1500 ppmw of iron. This significant improvement in methane conversion is unexpected especially when Comparative Examples B and C are compared as Comparative Example B has 500 ppmw less iron than Comparative Example C, but has better methane conversion indicating that increasing iron content does not always lead to a predictable increase in methane conversion.

Table 1 also indicates that while increasing iron composition in the samples continued to improve methane conversion up to 100%, samples with high iron loading (i.e., Sample 4) have decreased propane conversion when compared to samples with lower iron loading (i.e., Samples 1-3). Samples with higher iron loading, however, still provide acceptable propane conversion and propylene selectivity. Catalysts with high iron loading, such as greater than or equal to 2300 ppmw of iron may have superior long-term performance when compared to catalysts with less iron loading, such as less than 2300 ppmw. This is because catalysts with lower iron loading may suffer a significantly greater decrease in catalytic activity over time during the dehydrogenation process when compared to catalysts with higher iron loading. This means that it is ideal to maximize iron loading while maintaining sufficient dehydrogenation product selectivity. Table 1 indicates that Samples with iron loading from 2300 ppmw to 30000 ppmw maximize iron loading while maintaining sufficient propylene selectivity.

Further, Table 1 indicates that samples without both platinum and gallium (i.e., Comparative Examples D-H) have lower propane conversion and propylene selectivity than samples with both platinum and gallium (i.e., Samples 1-4). That is, Table 1 further indicates that the overall catalyst composition of platinum, gallium, and iron, is important for improving methane conversion while maintaining acceptable propane conversion and propylene selectivity.

### Example 2

### Effect of Sequential Impregnation

In Example 2, a sample of catalytically active particles (i.e., catalysts) was prepared and the effect of sequential impregnation on catalytically active particle's dehydrogenation and combustion performance was examined. The sample in Example 2 was prepared by using a base catalyst (i.e., Sample A) prepared using the procedure in Example 1. This pre-made catalyst was then promoted by impregnating with iron (III) nitrate solution in DI water followed by drying at a temperature less than 200° C., and then calcination at a temperature less than 800° C. The composition of the sample and its dehydrogenation and combustion performances is reported in Table 2.

TABLE 2

| | | | | | | | |
|---|---|---|---|---|---|---|---|
| | | | | | Propane | Propylene | Methane |
| | Pt | Ga | K | Fe | Conversion | Selectivity | Conversion |
| Sample | [ppm] | [wt. %] | [wt. %] | [ppm] | [%] | [%] | [%] |

| | | | | |
|---|---|---|---|---|
| 5 | Catalyst A + 3000 ppm Fe | 43.1 | 99.0 | 94.6 |

As Table 2 indicates, the improvement in methane conversion for compositions having iron in an amount from 2300 ppmw to 30000 ppmw occurs even when the catalyst is prepared using sequential impregnation. Sample 5 has significantly improved methane conversion when compared to Comparative Example C, which has only 1500 ppmw of iron. This means that the catalyst can be prepared using either sequential impregnation or co-impregnation.

### Example 3

### Effect of Order of Sequential Impregnation

In Example 3, 2 different samples of catalytically active particles (i.e., catalysts) were prepared and the effect of sequential impregnation on catalytically active particle's dehydrogenation and combustion performance were examined. The samples in Example 3 were prepared by using sequential impregnation to load the metals followed by drying at a temperature less than 200° C., and then calcination at a temperature less than 800° C. after each impregnation step. The composition of the samples, the order of their impregnation, and their dehydrogenation and combustion performances are reported in Table 3.

TABLE 3

| | | | | | | Propane | Propylene | Methane |
|---|---|---|---|---|---|---|---|---|
| | | Pt | Ga | K | Fe | Conversion | Selectivity | Conversion |
| Sample | Prep method | [ppm] | [wt. %] | [wt. %] | [ppm] | [%] | [%] | [%] |
| 4 | Co-impregnation | 300 | 1.6 | 0.25 | 10000 | 27.8 | 97.0 | 100 |
| 6 | Sequential | 300 | 1.6 | 0.25 | 10000 | 27.3 | 97.6 | 100 |
| | 1^{st} impregnation: | | | | | | | |
| | Pt/Ga/K | | | | | | | |
| | 2^{nd} impregnation: | | | | | | | |
| | Fe | | | | | | | |
| 7 | Sequential | 300 | 1.6 | 0.25 | 10000 | 25.1 | 97.7 | 100 |
| | 1^{st} impregnation: | | | | | | | |
| | Fe | | | | | | | |
| | 2^{nd} impregnation: | | | | | | | |
| | Pt/Ga/K | | | | | | | |

As Table 3 indicates, samples prepared by sequential impregnation (i.e., Samples 6 and 7) did not have a significant difference in their methane conversion, propane conversion, or propylene selectivity when compared to a sample prepared by co-impregnation (i.e., Sample 4). That is, the catalyst can be prepared using either sequential impregnation or co-impregnation.

### Example 4

### Effect of Gallium and Platinum Composition on Catalyst Performance

In Example 4, 3 different samples of catalytically active particles (i.e., catalysts) were prepared and the effect various properties, such as varying compositions, of catalytically active particles have on dehydrogenation and combustion activity were examined. The samples in Example 4 were prepared using the procedure of Example 1. The composition of the samples and their dehydrogenation and combustion performances are reported in Table 4.

TABLE 4

| | | | | | Propane | Propylene | Methane |
|---|---|---|---|---|---|---|---|
| | Pt | Ga | K | Fe | Conversion | Selectivity | Conversion |
| Sample | [ppm] | [wt. %] | [wt. %] | [ppm] | [%] | [%] | [%] |
| 8 | 10 | 0.4 | 0.25 | 3000 | 35.4 | 98.9 | 92.7 |
| 9 | 80 | 3 | 0.25 | 4500 | 49.2 | 98.7 | 96.0 |
| 10 | 300 | 0.4 | 0.1 | 3000 | 43.5 | 99.2 | 98.2 |

As Table 4 indicates, samples with iron in an amount from 2300 ppmw to 30000 ppmw have improved methane conversion with acceptable propane conversion and propylene selectivity across a range of platinum and gallium compositions. For example, Sample 9 has improved methane conversion, propane conversion, and propylene selectivity when compared to Comparative Example A even though Sample 9 has less platinum and more gallium than Comparative Example A. All samples in Table 4 (i.e., Samples 8-10) have improved methane conversion compared to Comparative Example A.

### Example 5

### Effect of Fuel Used During Combustion

In Example 5, 3 different samples of catalytically active particles (i.e., catalysts) were prepared and the effect fuel composition during the combustion step of the dehydrogenation and regeneration testing cycles has on catalytically active particles dehydrogenation and combustion activity was examined. In Example 5, the catalytically active particles were prepared using the procedure in Example 1. They were then run through dehydrogenation and regeneration testing cycles as described in Example 1, but the combustion step was changed to be combustion at 730° C. under 2.5 mol % H2/balance air (100% H2 fuel) with a flow rate of 50 sccm for 3 minutes. Combustion data was still collected at 60 seconds time on stream as in the previous examples. The composition of the samples and their dehydrogenation and combustion performances are reported in Table 5.

TABLE 5

| | | | | | | Propane | Propylene | Fuel |
|---|---|---|---|---|---|---|---|---|
| | Pt | Ga | K | Fe | Fuel | Conversion | Selectivity | Conversion |
| Sample | [ppm] | [wt. %] | [wt. %] | [ppm] | Used | [%] | [%] | [%] |
| A | 300 | 1.6 | 0.25 | 0 | H₂ | 41.3 | 98.7 | 100 |
| 1 | 300 | 1.6 | 0.25 | 3000 | | 49.2 | 99.1 | 100 |
| 2 | 300 | 1.6 | 0.25 | 5000 | | 44.3 | 98.9 | 100 |
| A | 300 | 1.6 | 0.25 | 0 | CH₄ | 38.9 | 98.4 | 80.9 |
| 1 | 300 | 1.6 | 0.25 | 3000 | | 50.2 | 98.9 | 95.4 |
| 2 | 300 | 1.6 | 0.25 | 5000 | | 45.0 | 98.8 | 99.2 |

As Table 5 indicates, samples with iron in an amount from 2300 ppmw to 30000 ppmw (i.e., Samples 1 and 2) have improved propane conversion and propylene selectivity when compared to samples without iron (i.e., Comparative Example A) even when the process is run without the use of methane as a supplemental fuel. Samples with iron in an amount from 2300 to 30000 ppmw (i.e., Samples 1 and 2) have the most significant improvement in fuel conversion and in propane conversion, when compared to Comparative Example A, in the presence of methane fuel, but still have some improvement in propane conversion when compared to Comparative Example A when methane is not used as a fuel.

In a first aspect of the present disclosure, a catalyst may comprise from 0.1 wt. % to 10 wt. % of gallium, from 5 ppmw to 1000 ppmw of platinum, from 2300 ppmw to 30000 ppmw of iron, and at least 85 wt. % support. Where the support may comprise one or more of alumina, silica, or combinations thereof.

A second aspect of the present disclosure may include the first aspect where the catalyst comprises from 10 ppmw to 400 ppmw of platinum.

A third aspect of the present disclosure may include any of the previous aspects where the catalyst comprises from 0.1 wt. % to 5 wt. % of gallium.

A fourth aspect of the present disclosure may include any of the previous aspects where the catalyst comprises from 2300 ppmw to 10000 ppmw of iron.

A fifth aspect of the present disclosure may include any of the previous aspects where the catalyst further comprises 0.01 wt. % to 2.5 wt. % of one or more alkali or alkaline earth metals.

A sixth aspect of the present disclosure may include the first aspect where the catalyst further comprises 0.01 wt. % to 2.5 wt. % of potassium.

A seventh aspect of the present disclosure may include any of the previous aspects where the catalyst comprises from 10 ppmw to 400 ppmw of platinum, from 0.1 wt. % to 5 wt. % gallium, and from 2300 ppmw to 8000 ppmw of iron.

An eighth aspect of the present disclosure may include the first aspect where the catalyst consists essentially of from 5 ppmw to 1000 ppmw of platinum, from 0.1 wt. % to 10 wt. % of gallium, from 2300 ppmw to 30000 ppmw of iron, and at least 85 wt. % support. Where the support comprises one or more of alumina, silica, or combinations thereof.

A ninth aspect of the present disclosure may include the first aspect where the catalyst consists essentially of from 10 ppmw to 400 ppmw of platinum, from 0.1 wt. % to 5 wt. % gallium, from 2300 ppmw to 8000 ppmw of iron, and at least 85 wt. % support. Where the support comprises one or more of alumina, silica, or combinations thereof.

A tenth aspect of the present disclosure may include the first aspect where the catalyst consists of from 5 ppmw to 1000 ppmw of platinum, from 0.1 wt. % to 10 wt. % of gallium, from 2300 ppmw to 30000 ppmw of iron, and at least 85 wt. % support. Where the support comprises one or more of alumina, silica, or combinations thereof.

An eleventh aspect of the present disclosure may include the first aspect where the catalyst consists of from 10 ppmw to 400 ppmw of platinum, from 0.1 wt. % to 5 wt. % gallium, from 2300 ppmw to 8000 ppmw of iron, and at least 85 wt. % support. Where the support comprises one or more of alumina, silica, or combinations thereof.

A twelfth aspect of the present disclosure may include any of the previous aspects where the catalyst has Geldart group A or Geldart group B properties.

A thirteenth aspect of the present disclosure may include a method of making a catalyst comprising impregnating the support with gallium and platinum to create a gallium and platinum impregnated support, drying the gallium and platinum impregnated support, calcining the gallium and platinum impregnated support, impregnating the gallium and platinum impregnated support with iron following the calcining of the support, and drying and calcining the impregnated support following impregnating the impregnated support with iron. Where the catalyst is the catalyst of the first aspect.

A fourteenth aspect of the present disclosure may include a method of making a catalyst comprising impregnating the support with iron to create an iron impregnated support, drying the iron impregnated support, calcining the iron impregnated support, impregnating the iron impregnated support with gallium and platinum following the calcining of the support, and drying and calcining the iron impregnated support following the impregnation with gallium and platinum. Where the catalyst is the catalyst of the first aspect.

A fifteenth aspect of the present disclosure may include a method of making a catalyst comprising impregnating the support with gallium, platinum, and iron, drying the support following the impregnating of the support, and calcining the support. Where the catalyst is the catalyst of the first aspect.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A catalyst suitable for making light olefins by dehydrogenation, the catalyst comprising:
from 5 ppmw to 1000 ppmw of platinum;
from 0.1 wt. % to 10 wt. % of gallium;
from 2300 ppmw to 30000 ppmw of iron; and
at least 85 wt. % support, wherein the support comprises one or more of alumina, silica, or combinations thereof.

2. The catalyst of claim 1, wherein the catalyst comprises from 10 ppmw to 400 ppmw of platinum.

3. The catalyst of any of the previous claims claim 1, wherein the catalyst comprises from 0.1 wt. % to 5 wt. % of gallium.

4. The catalyst of any of the previous claims claim 1, wherein the catalyst comprises from 2300 ppmw to 10000 ppmw of iron.

5. The catalyst of any of the previous claims claim 1, further comprising 0.01 wt. % to 2.5 wt. % of one or more alkali or alkaline earth metals.

6. The catalyst of claim 1, further comprising 0.01 wt. % to 2.5 wt. % of potassium.

7. The catalyst of any of the previous claims claim 1, wherein the catalyst comprises:
from 10 ppmw to 400 ppmw of platinum;
from 0.1 wt. % to 5 wt. % gallium; and
from 2300 ppmw to 8000 ppmw of iron.

8. The catalyst of claim 1, wherein the catalyst consists essentially of:
from 5 ppmw to 1000 ppmw of platinum;
from 0.1 wt. % to 10 wt. % of gallium;
from 2300 ppmw to 30000 ppmw of iron; and
at least 85 wt. % support, wherein the support comprises one or more of alumina, silica, or combinations thereof.

9. The catalyst of claim 1, wherein the catalyst consists essentially of:
from 10 ppmw to 400 ppmw of platinum;
from 0.1 wt. % to 5 wt. % gallium;
from 2300 ppmw to 8000 ppmw of iron; and
at least 85 wt. % support, wherein the support comprises one or more of alumina, silica, or combinations thereof.

10. The catalyst of claim 1, wherein the catalyst consists of:
from 5 ppmw to 1000 ppmw of platinum;
from 0.1 wt. % to 10 wt. % of gallium;
from 2300 ppmw to 30000 ppmw of iron; and
at least 85 wt. % support, wherein the support comprises one or more of alumina, silica, or combinations thereof.

11. The catalyst of claim 1, wherein the catalyst consists of:
from 10 ppmw to 400 ppmw of platinum;
from 0.1 wt. % to 5 wt. % gallium; and
from 2300 ppmw to 8000 ppmw of iron; and
at least 85 wt. % support, wherein the support comprises one or more of alumina, silica, or combinations thereof.

12. The catalyst of claim 1, wherein the catalyst has Geldart group A or Geldart group B properties.

13. A method for making a catalyst, the method comprising:
impregnating the support with gallium and platinum to create a gallium and platinum impregnated support;
drying the gallium and platinum impregnated support:
calcining the gallium and platinum impregnated support; and
impregnating the gallium and platinum impregnated support with iron following the calcining of the support;
drying and calcining the impregnated support following impregnating the impregnated support with iron;
wherein the catalyst is the catalyst of claim 1.

14. A method for making a catalyst, the method comprising:
impregnating the support with iron to create an iron impregnated support;
drying the iron impregnated support:
calcining the iron impregnated support; and
impregnating the iron impregnated support with gallium and platinum following the calcining of the support:
drying and calcining the iron impregnated support following the impregnation with gallium and platinum;
wherein the catalyst is the catalyst of claim 1.

15. A method for making a catalyst, the method comprising:
impregnating the support with gallium, platinum, and iron;
drying the support following the impregnating of the support; and
calcining the support; and
wherein the catalyst is the catalyst of claim 1.
